(19) United States
(12) Patent Application Publication
(10) Pub. No.: US 2012/0055472 A1
(43) Pub. Date: Mar. 8, 2012

(54) DOSE COUNTER

(75) Inventors: Lennart BRUNNBERG, Tyreso (SE); Henrik LANDAHL, Lund (SE)

(73) Assignee: LETCAT AKTIEBOLAG, Lund (SE)

(21) Appl. No.: 13/291,860

(22) Filed: Nov. 8, 2011

Related U.S. Application Data
(63) Continuation of application No. 11/920,874, filed on Nov. 21, 2007, now abandoned.
(60) Provisional application No. 60/683,778, filed on May 24, 2005.

(30) Foreign Application Priority Data
May 24, 2005 (EP) 05104409.7
Dec. 20, 2005 (SE) 0502812-1

(51) Int. Cl. A61M 15/00

(52) U.S. Cl. 128/203.12

(57) ABSTRACT

A dose counter device for an inhaler registers when a dose is delivered. The dose counter is adapted to mount on the distal end of the inhaler. The inhaler includes a canister comprising an inhaler housing. The dose counter device comprises a device trigger provided in an electron circuit on the distal end of the canister. The electron circuit further comprises an acoustic sensor also provided on the distal end of the canister. A force sensor is adapted to activate the acoustic sensor when a force is applied to the distal end of the canister. The acoustic sensor registers dose delivery when it picks up a sound.

This application is a continuation of U.S. application Ser. No. 11/920,874, filed on Nov. 21, 2007, which is the U.S. national phase of International Application No. PCT/SE2006/050159 filed 24 May 2006 which designated the U.S. and claims the benefit of U.S. Provisional Application No. 60/683,778, filed 24 May 2005; and claims priority to EP 05104409.7 filed 24 May 2005; and SE 0502812-1, filed 20 Dec. 2005, the entire contents of each of which are hereby incorporated by reference.

### INTRODUCTION

The technology described in this application relates to a dose counter device for an inhaler that in a reliable way registers a delivered dose from a canister comprised in the inhaler, and that at the same time substantially reduces the risk of falsely register a dose not delivered. Thus, the technology described in this application substantially avoids miscalculations of delivered doses from the canister.

### BACKGROUND

Within the field of inhalers, dose counters are known that will count the number of doses delivered from a canister comprised in the inhaler. The user will thus for instance know, the number of doses taken or the numbers of doses remaining in the canister. A problem with known dose counters, is that they at times will register a delivered dose that never was delivered, and that they also may miss to register a dose that in fact was delivered. The user of the inhaler is thus provided with false information about the number of doses remaining in the inhaler, which may constitute a major problem for instance an asthmatic person which thus unintentionally may run out of medicament.

A few solutions of mechanical dose counters have been presented during a number of years, such as for example EP-A2-0966309, wherein a dose counter is located near the valve region of the canister and attached to the base of an actuator, wherein the displacement of the top of the canister relative to the valve stem is measured.

In EP-A1-0254391, a dose counter is located on the top of the inhalation device, wherein the displacement of the top of the canister relative the actuator body is measured

Since canisters suffer from manufacturing height dimension variations and the counters in EP-A2-0966309 and in EP-A1-0254391 work taking into account the displacement of the canister, there exists a great risk of having counting errors.

There has also been presented a number of electrical and/or electronic solutions of dose counters. For example, GB 2288259 discloses a dose counter that comprises a button having a piezo-electric film sensor sensing the force applied to the button and the top of the canister when this is to be depressed for delivering a dose. When the force has reached a certain threshold value, a signal is sent to the electronic counter which registers a delivered dose.

GB 2398065 discloses a solution where an acoustic sensor in the form of a piezoelectric film is arranged on the canister at the outlet stem. The sensor registers the vibrations in the canister when a dose is delivered. The signal from the sensor is sent to a circuit at the top of the canister which registers a delivered dose.

The drawback with the solution according to GB 2288259 is that force measurement requires that the forces required to actually deliver a dose are held within a quite narrow range. The forces required depend on the spring in the canister and friction when pushing the stem into the canister, which can vary within a large range. This in turn means that the force sensed by the sensor may not be enough to deliver a dose so that the dose counter counts a dose even if none is delivered, or that the force required to deliver a dose is lower than the force level that is registered.

The drawback with the solution according to GB 2398065 is that the sensor is placed inside the inhaler close to the canister stem and that wiring is required between the sensor and the circuit at the top of the canister. It is thus rather difficult to arrange the dose counter to the canister, which could be done by an inexperienced user. If the sensor is not attached properly, the delivered doses will not be registered in a proper way, which in turn would lead to deviations between delivered number of doses and registered number of doses. Further, the placing of components inside the inhaler and thus in the inhalation airflow may affect the function of the inhaler in a negative way.

In view of the above, there is a general need for a dose counter device that in a reliable way will register a delivered dose from a canister comprised in the inhaler, and that at the same time substantially will reduce the risk of falsely register a dose not delivered. As understood, it is very difficult, if not impossible; to design a device that registers the in fact delivered dose with 100% accuracy. However, from the users' point of view, it is better to have at hand a dose counter device that occasionally may register a dose delivery even though no dose was in fact delivered, than to have it the other way around. In this way, the user can not unintentionally run out of medicament.

### SUMMARY

The aim of the technology described in this application is to remedy the above mentioned problems with accurate dose counters that are applicable to standard inhalers and standard canisters having differences in tolerances.

This aim is solved by the claimed inhaler 1.

A dose counter device for an inhaler registers when a dose is delivered and is adapted to be mounted on the distal end of the inhaler. The inhaler comprises a canister comprised in an inhaler housing. The distal end of the canister protrudes a distance from the distal end of the inhaler housing. The canister has a canister body and further in its proximal end has a dose chamber having a valve and a transfer tube having a valve. The valves are placed a predetermined distance from each other along the longitudinal axis of the canister when the inhaler is in a first non-activated state. The valves are adapted to communicate with each other when the distal end of the canister in a second activated state is applied with a force that urges the canister chamber over the transfer tube towards the proximal end of the canister a distance that is equal to the predetermined distance, which will expel medicament from the canister. The dose counter device comprises a device trigger provided in an electronic circuit on the distal end of the canister. The electronic circuit further comprises an acoustic sensor also provided on the distal end of the canister. The force sensor is adapted to activate the acoustic sensor when a force is applied to the distal end of the canister is substantially equal to and/or above a predetermined force value. The acoustic sensor registers a dose delivery when it picks up a sound.

According to another aspect, the device trigger is a contact and that the acoustic sensor is activated when the contact is closed due to the force applied to the distal end of the canister.

According to yet another aspect, the device trigger is a force sensor adapted to determine the force value applied to the distal end of the canister during the second activated state of the inhaler. The acoustic sensor is activated when the force that is applied to the distal end of the canister is substantially equal to and/or above a predetermined force value.

According to another aspect, the force sensor is a strain gauge or a piezo electric element.

According to a further aspect, the device trigger comprises both a contact and a force sensor. The closing of the contact activates the force sensor.

According to a further aspect, the acoustic sensor is a piezo electric element, a strain gauge or a microphone or the like.

According to yet another aspect, the force sensor is also used as the acoustic sensor.

According to a further aspect, the acoustic sensor is provided with means for comparing the spectra of a picked up sound with the spectra of the characteristic sound of a delivered dose and if there is a match between said spectra will register a sound as a dose delivery.

The technology described in this application has a number of advantages compared to the state of the art devices. One advantage is that both the device trigger and the acoustic sensor are placed at the distal end of the canister and thus the device. This means that it is easy for example to attach to the distal end of a canister of a press-and-breath inhaler and to use the device without having to adapt the inhaler to the device and/or to have components that have to be arranged inside the inhaler, which could be difficult for a patient to accomplish. Further, because no components need to be arranged in the interior of the inhaler, the device or its components will thus not affect the function or airflows through the device during inhalation. This may have the additional advantage that inhalers that have been approved by governmental authorities, such as the American FDA, do not need a further approval. The device is further easy and uncomplicated to manufacture.

Another advantage is that the acoustic sensor is only activated or triggered when a force is applied to the canister, i.e. when a dose is to be delivered. This means that the acoustic sensor cannot unintentionally register a sound, which may not be the sound of a delivered dose, i.e. it listens only during the time of dose delivery. Also, this greatly reduces the power consumption of the device.

One advantage, when the force sensor and the acoustic sensor are one and the same component, is that the number of components are reduced and thereby the manufacturing cost of the device is reduced. When a piezo electric element is used, it has the advantage that the power consumption is very low, and also that the device can be made very compact. Because of the properties of the piezo electric element, it may be used for other features such as sound generating, for example alerting a user that it is time to take a dose or to warn the user that there are only a few doses left in the canister.

The device could also have a “learning” ability, that it registers the sound spectra of delivered doses from a certain canister and then compares the sensed sound with the registered spectra. In that way the risk of wrongly sensing and registering sounds are further reduced. The learning ability could for example be done during the initial doses that are fired when a new canister is to be used. The learning ability means that the acoustic sensor will function with any type of canister regardless of substance, choice of material of the canister and the mechanics. Because of the learning ability and adaptive function the acoustic sensor will handle any possible change of the sound of a delivered dose during the life of the canister, which change of sound for example may be due to wear of components of the canister. It is also possible to have a rough “basic” reference spectra of a typical sound of a delivered dose stored in the electronic circuitry of the device, which basic spectra is used as the “starting” spectra for the acoustic sensing means to detect a delivered dose. During the initially fired doses the electronic circuitry modifies the basic spectra to the actual detected spectra.

In order to have a “double” security against unintended activation, the device trigger could comprise both a contact and a force sensor, such as a piezo electric element and arranged such that the force sensor is only activated when the contact breaker is closed, which is done when canister begins to be depressed. The closing of the contact also activates the rest of the circuitry of the device.

These and other aspects of and advantages will become apparent from the following detailed description and from the accompanying drawings.

### BRIEF DESCRIPTION OF THE DRAWINGS

FIG. 1 illustrates a general inhaler comprising a liquid medicament containing canister, when the inhaler is in a non-activated state,

FIG. 2 shows a cross-section of an electronic dose counter device to be used with a medical dispenser,

FIG. 3 shows a schematic block diagram of the dose counter of FIG. 2.

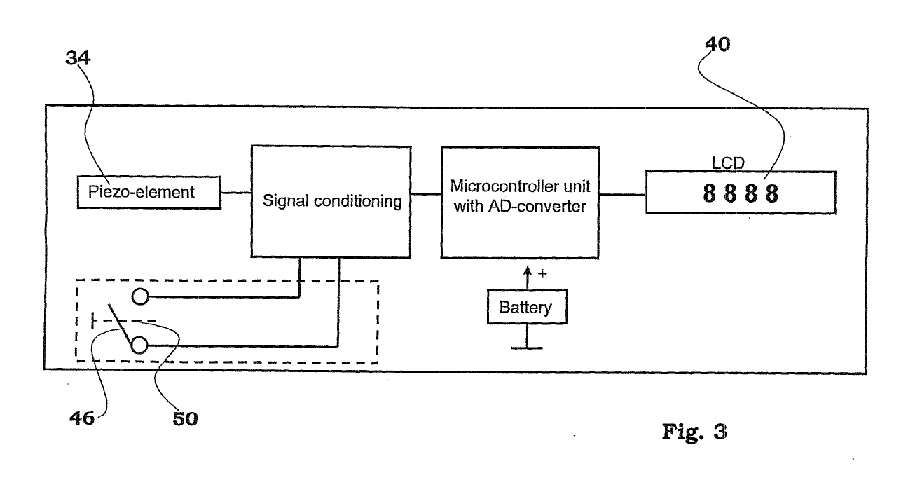

### DETAILED DESCRIPTION OF THE INVENTION

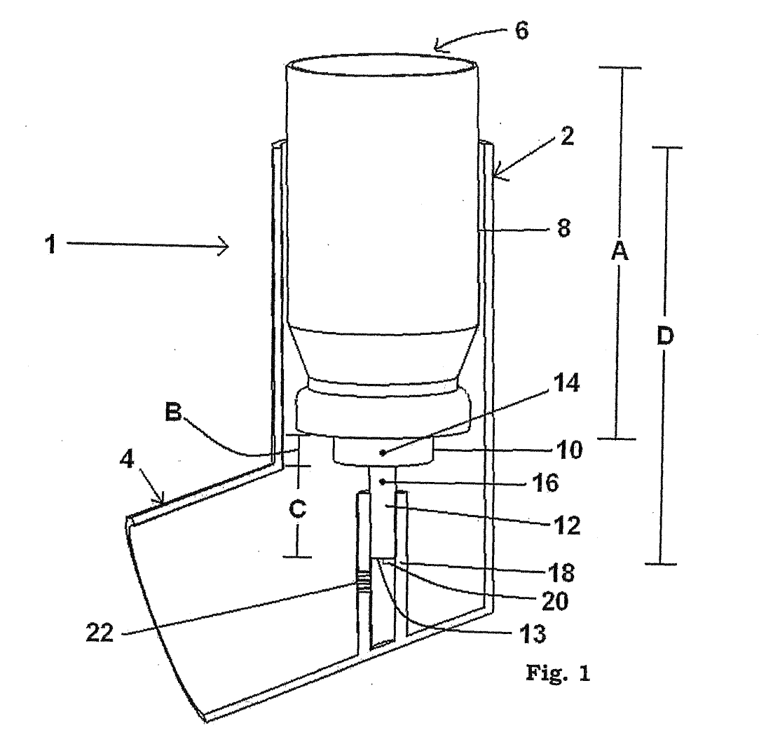

With reference to FIG. 1, a general inhaler 1 comprises a housing 2 having a mouthpiece 4, which the user puts in his mouth when a dose of medicament is to be inhaled. The housing 2 of the inhaler is adapted to receive a standard canister 6, containing liquid medicament, wherein the distal end of the canister 6 protrude a certain distance from the distal end of the housing 2.

The canister comprises a main canister body 8 that is adapted to communicate with a dose chamber 10. The dose chamber 10 is in turn provided with a hollow spring-suspended transfer tube 12 provided with an outlet 13 in its proximal end. The dose chamber is further provided with an outlet valve 14 that is adapted to correspond to a valve 16 in the transfer tube 12.

The interior of the mouthpiece 4 is provided with a tubular receiving member 18, having an inward protruding flange 20, provided a predetermined distance from the bottom of the receiving member 18. The receiving member 18 is further provided with an outlet 22 that communicates with the outlet of the mouthpiece 4.

The proximal end of the transfer tube 12 abuts against the flange 20, such that a part of the outlet 13 of the transfer tube communicates with the outlet 22 of the receiving member.

When a user of the inhaler intends to inhale a dose, he puts the mouthpiece in his mouth and applies a force, generally by the aid of his hand and fingers or the like, on the distal surface of the canister 6, such that the canister body 8 and the dose chamber 10 is forced downwards towards the bottom of the housing 2, i.e. towards the proximal end of the canister, while the transfer tube 12 remains still. Thus, when the dose chamber 10 has moved a predetermined distance towards the bottom of the housing, the valve 16 of the transfer tube 12 will open communication with the valve 14 of the dose chamber, such that a metered dose of the pressurised liquid medicament contained in the main body 8 will flow from the dose chamber 10, through the valves 14, 16, the transfer tube 12, the outlets 13, 22 and out through the outlet of the mouthpiece 4. When the user releases the force applied to the distal end of the canister, it will move back to its original position.

The distance between the valves 14, 16 is a known predetermined distance, generally 2 mm, when the inhaler is in the first non activated state, i.e. the dose chamber needs to in a second activated state be forced downwards with a distance of 2 mm for the valves 14, 16 to open communication with each other.

It would be a simple case to design a reliable dose counter device if the dimensions of the inhaler, would be exact dimensions. However, all dimensions of the general inhaler, such as the height of the main body A, the height of the dose chamber B, the length of the transfer tube C, and the distance between the proximal end of the transfer tube and the distal edge of the housing D, are impaired by variation of not negligible magnitude. If for instance the variation of the distances A, B, C and D is .+−.0.5 mm, .+−.0.05 mm, .+−.0.25 mm and .+−.0.2 mm, respectively, the sum of all margin of errors will be .+−.1 mm.

So, if one for instance designs a dose counter device that determines the distance that the canister has moved towards the bottom of the housing, dependent on a single reference point, for instance the distal edge of the housing 2, and register a delivered dose when said distance amount to 2 mm, the dose may or may not have been delivered. Also, the dose may have been delivered without the distance amounting to 2 mm.

It would be a simple case to design a reliable dose counter device if the dimensions of the inhaler, would be exact dimensions. However, all dimensions of the general inhaler, such as the height of the main body A, the height of the dose chamber B, the length of the transfer tube C, and the distance between the proximal end of the transfer tube and the distal edge of the housing D, are impaired by variation of not negligible magnitude. If for instance the variation of the distances A, B, C and D is .+−.0.5 mm, .+−.0.05 mm, .+−.0.25 mm and .+−.0.2 mm, respectively, the sum of all margin of errors will be .+−.1 mm.

Surrounding the bottom enclosure a top enclosure 36 is arranged, which is movable in the vertical direction of the device and the bottom enclosure 30 against the force of a spring 38. On the top of the top enclosure a display 40 is arranged, for example an LCD display. This in turn is connected to a printed circuit board (PCB) 42 comprising electronic circuitry which may for example comprise processors, I/O, and other applicable components for handling the function of the device, as will be described in detail below. The top enclosure is further arranged with a downwardly protruding arm 46 or protrusion.

The piezo electric element acts as a device trigger and may also have a double function as realized below, both as a force sensor and an acoustic sensor. When the patient or user depresses the top enclosure for delivering a dose of medicament the protrusion 46 will apply a pressure or force on the bottom enclosure 30. This force will cause the central part of the bottom wall of the bottom enclosure to bend due to that the bottom enclosure is only in contact with the canister at its periphery. The bending of the bottom enclosure will affect the piezo electric element, and a voltage is generated in the piezo electric element. When the force value amounts to a certain value, for instance 10 N that in many cases and for many canisters corresponds to a movement of the canister body towards the proximal end of the canister with a distance of 1 mm, will activate the electronic circuit in that an electric voltage is generated by the piezo electric element. At this moment the electronic circuit will start to sample signals from the piezo electric element in that the element now acts as an acoustic sensor, i.e. the element “listens” for sound. The delivery of a dose from the canister will namely generate a sound that will propagate to the distal end of the canister and which can be registered by the piezo electric element as a delivered dose.

By the above solution the piezo electric element is only activated to listen and detect the sound of a delivered dose when a force is applied to the piezo electric element, for example above or equal to 10 N, i.e. The acoustic sensor is adapted to operate within a predetermined range. This will minimize the risk of the acoustic sensor registering a sound that is not originating from the delivery of a dose. When the force applied by the user amounts to a certain force, one can be certain that the user intends to inhale a dose and that the sound of the dose delivery is soon to follow. Moreover, when the inhaler is not in use, the electronic circuit is not closed and thus the acoustic sensor is not active. However the display is preferably activated so that the patient at all times can view the number of doses. However, very little energy is consumed.

Preferably the electronic circuit is provided with means, such as a signal interpreting means, so that the acoustic sensor is adapted to register a sound as a delivered dose, only when it picks up a sound that corresponds to the sound that has its origin from the delivery of a dose. The delivery of a dose from the canister, namely generates a characteristic sound that can be identifiable by means of the signal interpreting means. Said interpreting means can for instance be provided with means that compares the spectra of the picked up sound with the spectra of the sound of a delivered dose. If there is a mismatch between said spectra, a sound is not registered as a dose delivery since the sound picked up thus had its origin from something else.

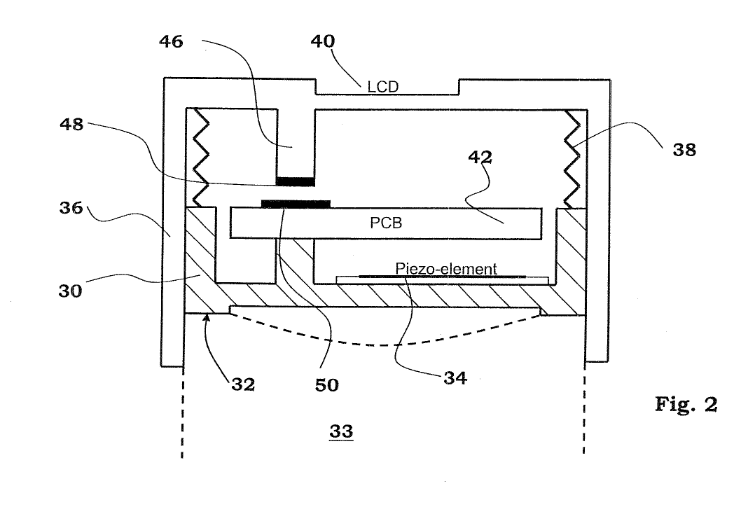

In order to further minimize the power need of the dose counter device, said device can be provided with an additional device trigger that closes the electronic circuit provided on the distal end of the canister only when a certain force is applied to said canister. This could for example, as shown in FIG. 2 be a conducting surface 48 arranged on the end of the protrusion 46, which conducting surface, when the top enclosure is depressed and the protrusion comes in contact with a contact surface 50 on the bottom enclosure, closes a circuit which activates the electronic circuit of the device. Only after closing the contact, the force sensor will be activated. The closing of the contact also activates the rest of the circuitry of the device.

The dose counter device is preferably provided with means in order to be connected to an external power source, such as a battery, even though it might be possible for the piezo electric element to be the only power source needed to operate the device.

It is of course feasible to use other components instead of a piezo electric element. For example a strain gauge could be used as the force sensor. In a simplified variant of the device, the device trigger could be just a contact and when the contact is closed, this activates the acoustic sensor to start to listen for the specific sound of a delivered dose. It is also conceivable that the acoustic sensor is a microphone and the like component that is capable of registering specific sound or vibration spectra, also spectra outside the audible spectra.

A further conceivable development of the device is to use “micro-mechanics”, i.e. to integrate several electrical and mechanical components in one or more chips, like for example acoustic sensing means, force sensor and other types of components and functions on a miniature bases.

Preferably the force sensor is also used as the acoustic sensor. For instance, if a strain gauge is used as the force sensor, a at least one further strain gauge can be provided on the distal end of the canister, which strain gauge is used as the acoustic sensing means as described above. It is also feasible that the strain gauge that serves as the force sensor, is adapted to also have the function of being the acoustic sensor. The same situation applies when the force sensor is a piezo electric element. That is, the piezo electric element used as the force sensor can also be used as the acoustic sensor, or at least one further piezo electric element, provided on the distal end of the canister, can be used as the acoustic sensor. Naturally, the use of a strain gauge as the force sensor does not rule out the use of a piezo electric element as the acoustic sensor, and vice versa. The components can thus be used in any combination. If a piezo electric element is used as the force sensor and/or the acoustic sensor, the dose counter device can be provided with a spring in order to reduce the flex of the piezo electric element in order to reduce of the risk for said element to break or crack.

The delivered dose and/or the doses remaining in the canister can be visualized for the user in a number of ways, such as through an electronic display provided in the inhaler or the like. Information about taken or remaining doses and e.g. time point may also be distributed by e.g. radiofrequency such as Bluetooth to another device where the information is displayed or used for compliance measuring, as described for instance in SE0300729-1.

Moreover, when a piezo electric element is used, because of its properties, it can be used to produce sound for example to alert a user at certain time intervals to take a dose of medicament or to warn the user that for example only ten doses remain and that the user soon should replace the canister with a new. In that aspect the circuit is pre-programmed with the total number of doses in a canister and when registering delivered doses counts down and displays the remaining number of doses. According to another aspect, the electronic circuit could be configured so that the registered sounds from the canister when the first two or three doses are delivered are stored and compared in order for the circuit to “learn” the specific spectrum of that canister, in order to increase the reliability that only the sounds of a delivered dose is registered.

The electronic circuit could also be configured to register and monitor the delivery of doses. For example, the electronic circuit could be provided with temperature sensors for measuring and storing the actual temperature at the time a dose was delivered. The circuitry then has to be added with a real-time clock for keeping track of time. It may also be configured to detect and monitor the air flow during inhalation, for measuring and storing the air flows at dose delivery. The acoustic sensor could also be able to listen to the specific sounds connected to inhalation. In that aspect, the learning function could be used as well as the comparison between the detected spectra and previously stored spectres.

Further, accelerometers could be provided for acting as shaking sensors for registering if the device has been shaken before use. All the information from these features could be used to register if the patient has been able to receive the doses properly, how the conditions during dose delivery were, i.e. to obtain a dose delivery history, so that a physician can advice its patient, and to maybe change the behaviour of the patient regarding handling of the device, change the frequency of delivered doses and the like.

It is to be understood that the embodiments described above and shown in the drawings are to be regarded only as non-limiting examples that may be modified in many ways within the scope of the patent claims.

1. A dose counter device for an inhaler that registers when a dose is delivered, adapted to be mounted on the distal end of the inhaler, the inhaler comprising a canister comprised in an inhaler housing, wherein the distal end of the canister protrudes a distance from the distal end of the inhaler housing, and wherein the canister has a canister body and further in its proximal end has a dose chamber having a valve and a transfer tube having a valve, wherein the valves are placed a predetermined distance from each other along the longitudinal axis of the canister when the inhaler is in a first non-activated state, and wherein the valves are adapted to communicate with each other when the distal end of the canister in a second activated state is applied with a force that urges the dose chamber over the transfer tube towards the proximal end of the canister a distance that is equal to the predetermined distance, which will expel medicament from the canister, wherein the dose counter device comprises a device trigger provided in an electronic circuit on the distal end of the canister, that the electronic circuit further comprises an acoustic sensor also provided on the distal end of the canister, and a force sensor adapted to activate the acoustic sensor when a force is applied to the distal end of the canister and in that the acoustic sensor registers a dose delivery when it picks up a sound.

2. The dose counter device according to claim 1, wherein the device trigger is a circuit closer, and wherein the acoustic sensor is activated when the circuit closer is closed due to the force applied to the distal end of the canister.

3. The dose counter device according to claim 1, wherein the device trigger is a force sensor, wherein the force sensor is adapted to determine a force value applied to the distal end of the canister during the second activated state of the inhaler, and wherein the acoustic sensor is activated when the force that is applied to the distal end of the canister is substantially equal to and above a predetermined force value.

4. The dose counter device according to claim 1, wherein the force sensor is a strain gauge or a piezoelectric element.

5. The dose counter device according to claim 1, wherein the device trigger comprises both a contact and a force sensor, and wherein the closing of the contact activates the force sensor.

6. The dose counter device according to claim 1, wherein the acoustic sensor is a piezoelectric element, a strain gauge, or a microphone.

7. The dose counter device according to claim 1, wherein the force sensor is also used as the acoustic sensor.

8. The dose counter device according to claim 1, wherein the acoustic sensor includes means for comparing the spectra of a picked up sound with the spectra of the characteristic sound of a delivered dose, and if there is a match between the spectra, for registering a sound as a dose delivery.
